(19) United States
(12) Patent Application Publication
(10) Pub. No.: US 2012/0078132 A1
(43) Pub. Date: Mar. 29, 2012

(54) Method and Apparatus of Neural Signal Determination and Therapy Enhancement

(76) Inventors: Mark Zdeblick, Portola Valley, CA (US); Robert White, Stanford, CA (US)

(21) Appl. No.: 13/319,974

(22) PCT Filed: May 19, 2010
(86) PCT No.: PCT/US10/35466

§ 371 Date: Dec. 13, 2011

Related U.S. Application Data
(60) Provisional application No. 61/180,116, filed on May 20, 2009.
(60) Provisional application No. 61/180,299, filed on May 21, 2009.

(51) Int. Cl. A61N 1/372; A61B 5/053

(52) U.S. Cl. 600/547; 607/62

(57) ABSTRACT

A method and device are provided that detect and identify a neural signal by type and optionally or automatically provide a signal that affects a nerve carrying the neural signal. A plurality of electrodes is positioned along a nerve and monitors the electrical activity of the nerve. The invented system analyzes the monitored data to determine when the monitored activity can be identified as a known type of bioelectric signal, e.g., a pain nerve signal. The system may optionally attempt to affect, disrupt, diminish, nullify or block transmission of bioelectric signals along the nerve, e.g., by directing electrical energy through an electrode to nullify a bioelectric signal along a pain nerve.

### CROSS-REFERENCE TO RELATED APPLICATIONS

Pursuant to 35 U.S.C. §119 (e), this application claims priority to the filing dates of U.S. Provisional Patent Application Ser. Nos. 61/180,116 (filed May 20, 2009) and 61/180,299 (filed May 21, 2009), the disclosures of which are herein incorporated by reference in their entireties.

### FIELD OF THE INVENTION

The present invention relates to bioelectric signal detection and measurement. More particularly, the present invention relates to therapeutic uses of bioelectric signal analysis.

### INTRODUCTION

The nervous system is a complex, sophisticated system that regulates and coordinates basic functions and activities. The nervous system includes two major divisions, namely the central nervous system (CNS) and the peripheral nervous system. The CNS includes the brain and spinal cord. The peripheral nervous system consists of the neurons throughout the body with the exception of those in the CNS. The neurons of the peripheral nervous system include the sensory neurons, which detect any sensory stimuli and alert the central nervous system of their presence, and motor neurons, which connect the central nervous system to the muscles and carry out instructions from the central nervous system for movement.

Diseases and disorders of the nervous system may significantly impact health and quality of life. Such diseases and disorders include brain aneurism, spinal cord injury, epilepsy, and Parkinson's disease. Major organs, systems, and body functionality can be adversely affected. Examples of the detrimental or undesirable impact of neural diseases and disorders include pain, tremor, loss of coordination and sensory functionality.

Consequently, various methods and apparatuses have been made available in attempting to treat such diseases and disorders. Examples include various devices and methods associated with neural signal generation and flow. Such attempts, however, have sometimes resulted in an inability to detect certain signals, to differentiate between discrete categories of signals, and/or to derive from such indicators useful information to effect desired treatment outcomes. Furthermore, the prior art fails to optimally provide for the detection and disruption of undesired activity by nerves, e.g., pain nerve activity.

Thus, there is continued interest in the development of effective, efficient treatments for, as well as management of, such diseases, disorders, and symptoms.

### SUMMARY

Toward this and other objects that are made obvious in light of the disclosure, a method and apparatus are provided for neural signal determination and therapy enhancement. According to a first aspect of the method of the present invention, a lead containing a plurality of electrodes are positioned with a living human body, or other living body, and along a nerve. The electrodes detect bioelectric signals and optionally provide electrical energy to the nerve that disrupts, affects, diminishes or nullifies certain types of bioelectric signals, e.g., signals along a pain nerve.

According to a second aspect of the method of the present invention, an additional factor relating to the living being, such as an ingestion of a medication, partly determines the actual and preferred application of the electrodes.

### BRIEF DESCRIPTION OF THE FIGURES

FIG. 1 illustrates a neural environment of a living being, or host, including a neural signal determination device;

FIG. 2 illustrates an exemplary pattern of neural signals;

FIG. 3 illustrates a pattern of neural signals found in the neural environment of FIG. 1;

FIG. 4A is a schematic of an exemplary satellite of the device of FIG. 1, wherein the satellite comprises at least two electrodes and an integrated circuit;

FIG. 4B is a schematic of a controller, or can, associated with the device;

FIG. 5 is a schematic of an ingestible event marker;

FIG. 6 is a schematic of a computer that receives wireless communications from the ingestible event marker of FIG. 5 and the controller of FIG. 4B;

FIG. 7 is an illustration of interactivity of the ingestible event marker of FIG. 5, the can of FIG. 4B, the computer and/or the clinician of FIG. 6;

FIG. 8 is a schematic of a diagnostic and therapeutic record stack are stored in the can system memory of FIG. 4B;

FIG. 9 is a flowchart of a first process of the device of FIG. 1, wherein a bioelectric signal is characterized and may be affected, e.g., diminished in energy or nullified, by the device of FIG. 1;

FIG. 10 is a flowchart of a second process wherein a human therapist at least partly determines the application of the device of FIG. 1;

FIG. 11 is a process chart of a method of modifying and storing a therapeutic action data of the diagnostic and therapeutic record stack of FIG. 8;

FIG. 12 is a process chart of a method of identifying and recording a digitized representation of a bioelectric energy state of the host of FIG. 7; and

FIG. 13 is a process chart of a method of improving the measurement and detection of an additional bioelectric signal that occurs simultaneously with the bioelectric energy state of FIG. 12.

### DESCRIPTION

It is to be understood that this invention is not limited to particular aspects of the present invention described, as such may, of course, vary. It is also to be understood that the terminology used herein is for the purpose of describing particular aspects only, and is not intended to be limiting, since the scope of the present invention will be limited only by the appended claims.

Methods recited herein may be carried out in any order of the recited events which is logically possible, as well as the recited order of events.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the methods and materials are now described.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

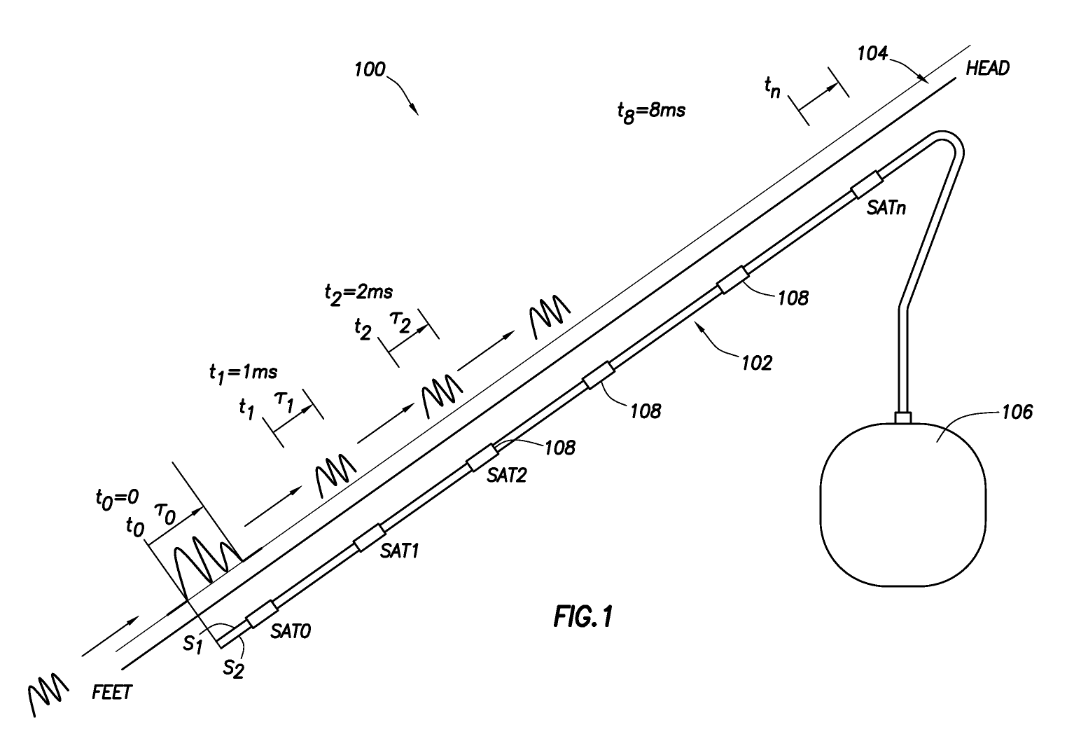

Referring now generally to the Figures and particularly to FIG. 1, FIG. 1 illustrates a neural environment 100, including a neural signal determination device 102, a portion of a nerve 104, and a controller module 106, e.g., integrated or otherwise associated with one or more devices, components, or subcomponents such as the can (hereinafter “the can 106”).

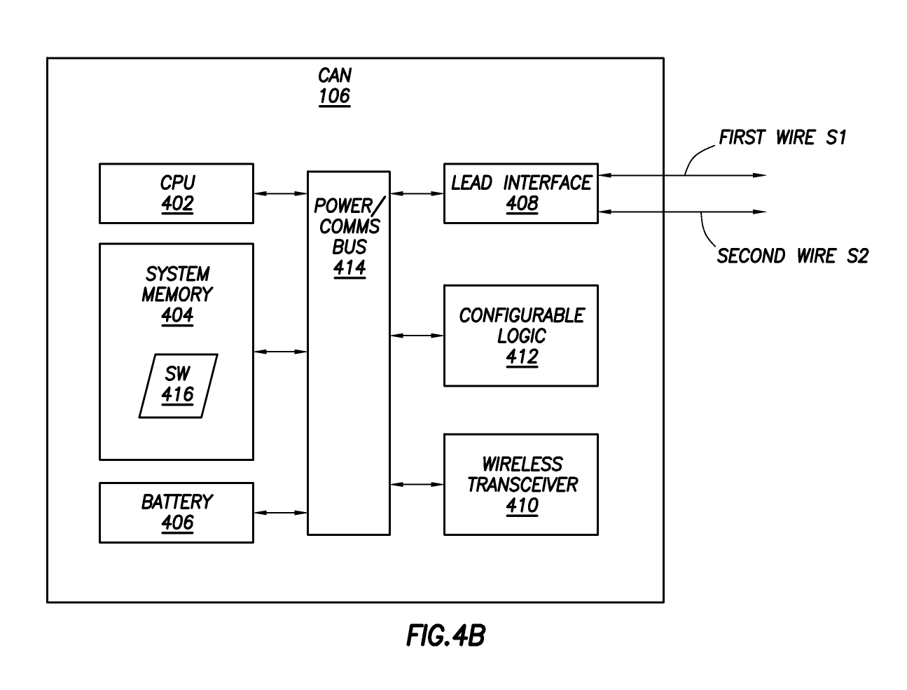

Generally, the invention provides the neural signal determination device 102 and a method for detecting discrete sets of signals associated with one or more nerves 104 and distinguishing signal directionality, e.g., afferent and efferent. Placement of a neural signal determination device 102, sometimes referred to herein simply as the “lead” 102 or catheter, and one or more satellites, e.g., sat 1, sat 2, etc., which include electrodes E1-EN, as shown in FIG. 4A, comprised within the lead 102 may exhibit electrical potential sensitivity to signals present at a discrete location, e.g., a location associated with, or very near to, a particular area of the nerve 104. Each electrode E1-EN is preferably within one centimeter of the nerve 104, and more preferably within one millimeter of the nerve, and even more preferably in contact with the dermal layer of the nerve 104. This electrical activity sensitivity may facilitate identification of signal patterns and/or characteristics sets, which may inform important treatment decisions and actions.

For example, sets of action potentials may be identified over time intervals. Identification of such sets may permit identification of directionality of signals, which in turn informs a variety of therapeutic-related decisions and actions. Included in the foregoing optional aspects of the method of the present invention are (a.) blocking a pain signal traveling to or from the brain to alleviate pain, (b.) stimulating along the path of the nerve 104 to facilitate normal signal functionality otherwise inhibited by disease, injury, and (c.) blocking a tremor signal travelling from the central nervous system. Other applications are also possible.

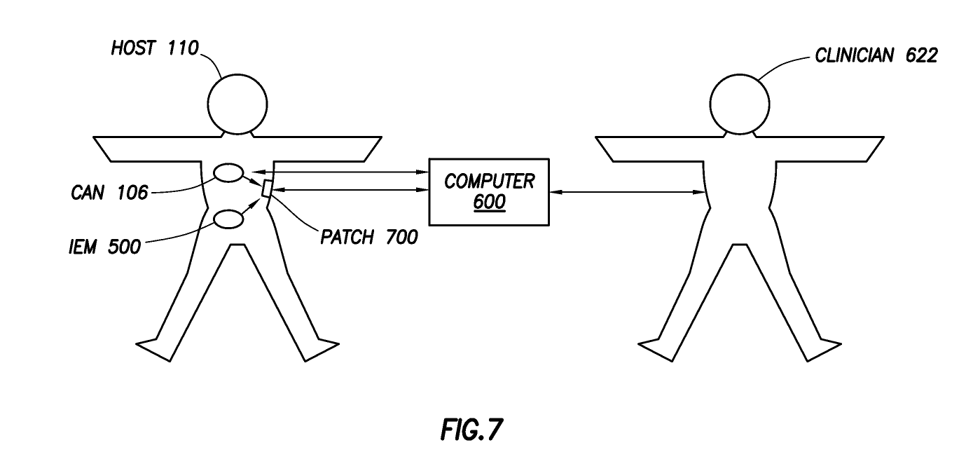

The neural environment 100 is typically associated with a living being 110, or host 110, as shown in FIG. 7, and generally includes at least a portion of one more nerves 104. The nerve 104 generally communicates signals in an afferent direction, e.g., toward a head 110A of the host 110, and in an efferent direction, e.g., toward a foot 1108 or hand of the host 110. Examples of nerves 104 include the vagus nerve, the spinal column, and the sciatic nerve.

In various aspects, the neural signal determination device 102 is associated with at least a portion of one or more nerves 104. The neural signal determination device 102 may be configured, for example, as a multiplexed lead having at least two electrical conductors or wires S1 and S2, and at least two electrodes E1 and E2, as shown in FIG. 4A. Variations of the lead 102 include multiplexing leads such as those disclosed U.S. Pat. No. 7,214,189 and U.S. patent application Ser. No. 10/734,490 published as 20040193021 (the disclosures of which patent and application are herein incorporated by reference), wherein such multiplexing leads may comprise at least one satellite 108, SAT1-SATn, wherein the at least one satellite 108 comprising at least two electrodes E1 and E2, e.g., a segmented electrode having two or more segments, such as two, four, five, six, eight segments, etc. In one example, the satellite(s) 108 may comprise a segmented electrode, such as a quadrant electrode. The satellite 108 may further comprise an integrated circuit 400 communicably associated with the at least two electrodes, as shown in FIG. 4A, and other components as heretofore disclosed. Of interest are satellites as described in U.S. Pat. No. 7,214,189 and U.S. patent application Ser. Nos. 11/793,904 published as 20080255647 and 11/794,016 published as 20080312726; the disclosures of the various satellites of these applications being herein incorporated by reference. One skilled in the art will recognize, however, that various neural signal determination devices 102 may be employed in conjunction with the present invention so long as the functionality described herein is carried out.

In various aspects, the neural signal determination device 102 may be associated, e.g., electrically communicate with, the can 106. The can 106 may be configured in various ways and provide various functionality, as is known in the art. Such functionality may include, for example, programming ability associated with configuring or programming the neural signal determination device 102.

More particularly, in various aspects the neural signal determination device 102 may be positioned proximal to at least a portion of the nerve 104 and/or along various alternate nerves. In various aspects, the electrodes E1-EN, pairs of electrodes, E1 and E2, E3 and E4, of the neural signal determination device 102 may be placed at regular spaced intervals or predetermined points relative to the nerve 104.

In one configuration, for example, the neural signal determination device 102 includes satellites 108 with segmented electrodes E1-E4 that are configured as quadrant electrodes E1-E4. The satellites 108 may be multiplexed to the two electrical conductors S1 and S2 along the neural signal determination device 102 such that two of the quadrant electrodes E1 and E2 can be attached to one of those wires, S1 and the other two quadrant electrodes E3 and E4 can be electronically connected to the second wire S2. This configuration may provide for a very fine aperture so that the quadrant electrodes E1-E4 are sensitive only to the action potentials that are very proximal to the neural signal determination device 102. This placement of the quadrant electrodes E1-E4 avoids the problems associated with picking up signals in a great distance and a great direction, which may inhibit accurate signal determination.

Continuing with the foregoing, a nerve propagation pattern that travels as a function of time toward the head 110A of the host 110 at t0 and, some time later, at t1 may be recorded at predetermined times and for predetermined durations of time. For example, recording action potentials at a first site of a first satellite SAT1, e.g., sat 0, starts and recording continues for a period of time, e.g., t1, shows up as a time-varying electrical signal that is picked up by a second satellite SAT2 at the distal end of the neural signal determination device 102. If recording of action potentials start at t1 is communication via S1 and S2 to the can 106, an aspect of the present invention includes an initially programmed neural signal determination device 102 such that half of the electrodes E1 and E2 of the first satellite SAT0 are connected to one of the wires S1 and the other half of the electrodes E3 and E4, are connected to S2. One skilled in the art will recognize that other configurations of electrodes, e.g., two quadrants, may be used. Alternatively, the neural signal determination device 102 may be programmed, e.g., at the outset of performing the method of the present invention.

At t0 and for T0, recording begins inside the can 106 of the differential potential between the two electrodes E1 and E2. The difference of potential of the two electrodes E1 and E2 is measured and, because each of the two electrodes E1 and E2 is so close to the other, the two electrodes will only be sensitive to electrical signals that are very close to the two electrodes E1 and E2. Stated differently, the two electrodes E1 and E2 are sensitive only to electric signals generated in very close proximity, such as the signals of the nerve bundle very near the two electrodes E1 and E2. Recording is continued for a period of time, approximately one half of a millisecond, approximately one millisecond, a microsecond, or for any other period of time, as so determined. Once the recording period has elapsed, the recording functionality is directed to the next two electrodes, e.g., the second satellite SAT1, in the set of electrodes E1-EN configured on the neural signal determination device 102. At the first satellite SAT1, recording commences at t1 for a period of T1. It is noted that various time intervals, e.g., T0, T1, T2, etc., may be of the same or differing durations.

Generally, in the time taken between t0 and t1, the first action potential that was recorded travelled up the nerve 104 to the second action potential in which time it was measured again and then, similarly, at the second satellite SAT1 and at time t2, the recording commences for a period of T2 and that action potential travels again a distance along the nerve 104 and shows up at the second satellite SAT1. In this manner, if the velocity of an action potential travelling up the electrodes E1-EN is known or derived, then the action potential shows up in the same time after the beginning of each of these electrodes. This pattern can continue on n number of electrodes E1-En and the more electrodes E1-EN present and the more time periods used, the better the ultimate resolution of separating out the directionality, e.g., afferent and efferent.

In various aspects, the method is performed in the can 106, in situations having n number of voltage patterns as a function of time and each at a certain delay of time from the previous one. If that delay is the same and the distance between the electrodes is the same, then the pattern that reappears precisely among all n of those will be those action potentials travelling at that exact velocity. For example, the neural signal determination device 102 is configured with eight satellites Sat0-SAT7. Starting at t0 and waiting exactly a millisecond to t1 and then measuring for a millisecond and then exactly a millisecond later (t2)—thus t=0, t=1 millisecond started at t2=2 millisecond and at t=7 8 milliseconds—each of those are approximately one millisecond long in recordings. All eight measurements, one measurement from each of the eight satellites SAT0-SAT7, averaged together provides results where the action potentials that are travelling towards the head at the rate of one satellite distance per millisecond (and if the satellites are one cm apart) that would represent those action potentials travelling towards the head that are travelling at the rate of one cm per millisecond.

Thus, the invention may also be employed to determine which type of signals travel quickly in a region, and whether or not those nerves areas tend to amplify or accelerate rate of travel.

More particularly, if the velocity travelling in a given direction is adjusted, then the action potential of interest would show precisely at the same interval from the beginning of the recording. If the velocity is not gauged, e.g., if it is travelling faster than the sampling, it would tend to progress ahead. Thus, in addition to simply taking the average of the eight measurements, correlations of shifts of time between them may be done where each of those shifts of time represents a different velocity. Generally, certain aspects provide for sequences, e.g., from t0 to t8 using the eight satellite approach, then back to t0 and starting all over again, and building up such as sequence over time.

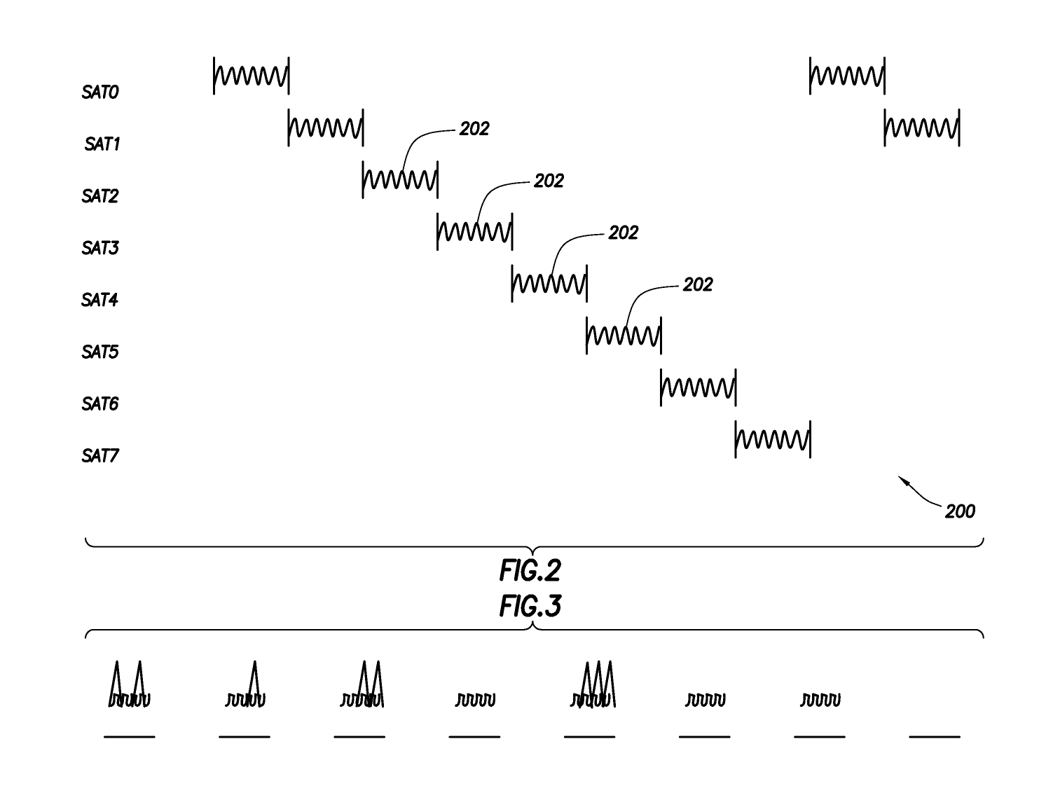

FIG. 2 illustrates an exemplary pattern 200 of neural signals 202. With respect to a zero satellite SAT0 and a time tracing the nth satellite SATn, the time trace0 will have some action potential for a period of time and then shortly thereafter, the time trace 1 will start and then time trace 2 and then probably continue to t4, e.g., times 0, 1, 2, 3, 4, 5, 6, and 7, and then go back to zero again and then to 1 again. This may permit determination of directionality, e.g., afferent directionality (in this example, moving in a direction toward the head) and comparing measurements of action potentials travelling in that direction. With respect to the opposite direction, the process may begin at SAT7 and sequentially move back towards SAT0, thus detecting efferent directionality, e.g., signals moving towards the feet. In this manner, the invention provides for directionality determination.

Summarizing, this method of sampling electrical potential measured by the satellites SAT0-SATn may be accomplished by generally providing a string of electrodes E1-EN positioned at locations associated with at least a portion of the nerve 104, each location of which has at least two electrodes E1-EN that are fairly close to the location so that the respective two electrodes E1-EN are sensitive only to those signals that are very nearby and can distinguish from various other signals.

Another use of the present invention is to employ the gathered information to effect a decision or action. To illustrate, if a health-related goal is to mask pain or provide stimulation to a predetermined area via the nerve, various aspects may utilize an electrode, e.g., a ninth electrode configured on the lead, that generates a signal according to predetermined parameters, e.g., at either an appropriate location along the nerve, at an appropriate time, or both. The predetermined parameters may be based on the information gathered from the aforedescribed method and apparatus.

To further illustrate, a certain pattern going towards a brain of the host 110 is identified. A particular signal of the pattern is recognized at a point in time and identified as a pain signal travelling toward the brain. A ninth electrode pair E17 & E18 stimulates at a precisely-recognized point in time (identification of the pain signal) to cancel out the pain signal before its arrival at the brain, thus alleviating a patient's pain.

In various aspects, various time frames may be used. Larger timeframes include, for example, milliseconds, microseconds, seconds and greater periods of time. At a predetermined point of time, e.g., based on patterns and/or information identified in the aforedescribed process, the lead 102 delivers electrical energy through the electrodes E1-En for a period of time to nullify or diminish bioelectric signals that are actually travelling at a predetermined rate. For example, a bioelectric signal travelling along the nerve 104 that would cause a sense of pain to be perceived by the host 110 if the instant bioelectric signal reached the brain of the host 110 may be nullified or significantly reduced by electrical energy released by one or more electrodes E1-EN.

According to yet another aspect of the method of the present invention, the lead 102 may have or employ twice as many electrodes E1-EN as previously posited. To illustrate, a lead 102 s configured with 16 electrodes E1-E16, where the first eight electrodes E1-E8 are used to sense those signals travelling toward the brain and the next eight electrodes E9-E6 are used to take the energy out of those signals travelling towards the brain, e.g., by stimulating with the lead 102 to decrease or cancel the bioelectric signals of the nerve 104. Alternatively, the first eight electrodes E1-E8 may be used to stimulate the nerve 104 to cancel a bioelectric signal.

In one use, the first group of electrodes E1-E8 may be employed to sense those bioelectric signals going towards the brain and the second group of electrodes E9-E16 may be used to pace a portion of electrical energy at a ninth electrode or ninth satellite SAT9 and then wait for a predetermined amount of time, for example, one millisecond. After which time the ninth satellite SAT9 is paced (at precisely a millisecond later), and again at a millisecond later, e.g., repeatedly at a given delay at each subsequent location of a satellite SAT10-SATn. Each of the satellites SAT10-SATn may expend an element of the electrical energy provided by the can 106 to the lead 102 and furthermore, each satellite SAT10-SATn may apply the electrical energy provided by the can 106 to nullify a portion of the electrical energy of the action potential of the bioelectric signal so that by the time the bioelectric signal gets to a particular electrode E1-EN, there is no biologically significant electrical energy left in the bioelectric signal. And then, via an exemplary sixteenth satellite SAT16, a determination can be made whether the electrical energy of the lead 102 has been completely expended by the electrodes E1-EN and that the electrical energy of the lead 102 has canceled the travelling bioelectric signal.

Still further, in cases where certain types of bioelectric signals travel faster than other types of bioelectric signals, e.g., pain signals, recording such velocities and paths of travel may enable identification by type of a bioelectric signal detected by the lead 102.

Various aspects include various neural signal determination devices. Some examples of alternate or additional aspects of the invented leads 102 include multiplexed lead device, hardwired-devices, e.g., devices having one electrode per wire, and other devices.

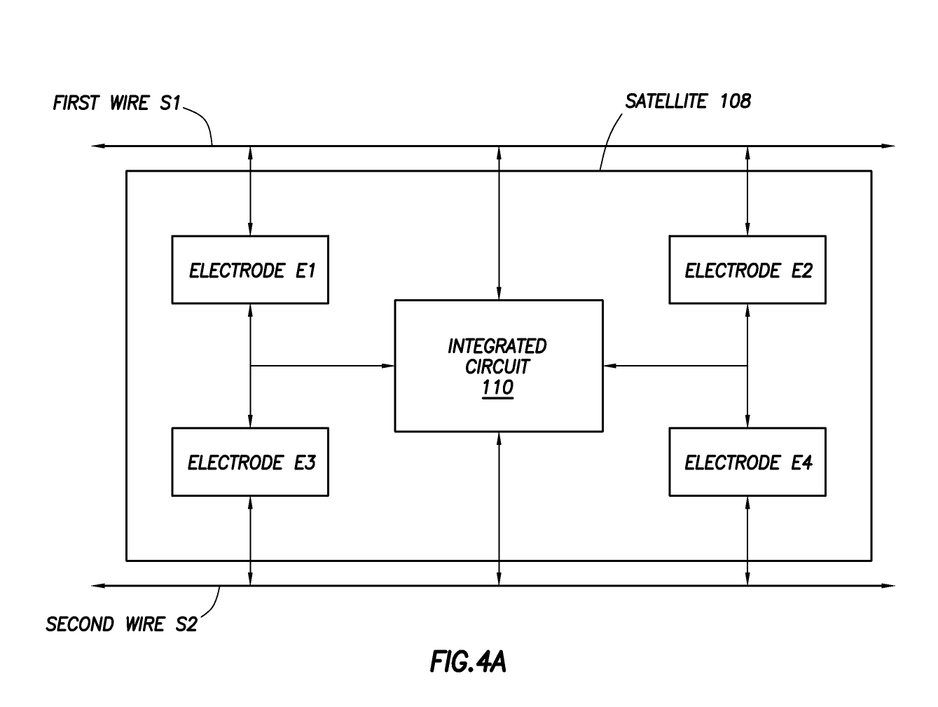

Further, various other aspects of the invented method provide a simple use for blocking pain or for measuring and detecting signals that are representative of some physiologic event that can be transmitted by nerves 104 to the brain. For example, a pattern of action potentials at a certain time of sequence may indicate that pain is a pattern (illustrated in FIG. 3 as a set of rapidly spaced pulses occurring at a time interval) representative of how fast signals travel in a direction, thus that pattern would signal pain. If stimulated at a location below the pattern location, an inference may be made whether or not the pain has been blocked by the absence or presence of the pain signal.

In various aspects, the invented method allows the gathering of data to inform treatment decisions, take various therapeutic actions by in part by determining qualities of the bioelectric signal. Sampling the bioelectric signal by the plurality of leads E1-EN at multiple locations of satellites SAT0-SATn and determining a wave form allows pattern identification of a detected bioelectric signal. In certain circumstances, if a single electrode is used, e.g., a ring electrode, a bioelectric signal be detected but confusion about the nature of the detected bioelectric signal may arise if the directionality of the bioelectric signal exists but not is not considered. For example, and with reference again to FIG. 3, there are different locations of satellite SAT0-SATn and at these electrode locations there is a potential that is sought, i.e., trying to pick out, there is something that shows up in a recurring way at each of the electrode locations. In addition, other information going in other directions so the opposite direction may show a signal that is here and there, which may show up as two signals, then one signal, or no signal may be detected by an electrode E1-EN at that particular point in time, then three signals show up, and so forth. Thus, the electrode measurements may be averaged, and the amplitude of an electrical potential measurement taken at a particular location of one satellite SAT0-SATn or one electrode E1-EN may be computationally reduced by division by the can 106, but the bioelectric signal measurements taken by one or more electrodes E1-EN may reinforce other bioelectric signals, tending to lead to an unreliable or inaccurate observation about the nature of a particular bioelectric signal.

Certain bioelectric signals may randomly show up and dissipate. Thus, the more the occurrence of the non-random signals, the better the resolution in terms of the bioelectric signal that is actually traveling at that point in time in that direction. However, employing the first wire S1 and/or the second wire S2 for each of the electrodes E1-EN may result in a very still lead 102 that is difficult to maneuver as well as create other issues such as corruption of signals and breaking of wires S1 and S2. Therefore, in some aspect, preferably a small number of wires, e.g., two, four, etc., are employed within the lead 102.

Further alternative aspects of the present invention include the ability of having one satellite SAT0-SATn opposite a neighboring satellite SAT0-SATn and positioning the first two wires S1 and S2 to go back to the can 106 and connect every other satellite SAT0-SATn that is connected to one of the two wires S1 and S2. This may be employed, for example, to facilitate overlap in the time domains.

In various aspects, a small number of wires S1 and S2, e.g., two, four, five, six, etc., may be employed with a variety of different techniques used between multiplexing and gathering overlapping sets of information at different points in time. In one example, if the velocity of a bioelectric signal of interest is not known or if the velocity of the instant signal is fast, this aspect may facilitate overlapping time windows to correlate between the fast-travelling signals.

In various other alternate aspect of the invented method, having a small number of wires S1 and S2, such as those configured with a large number of satellites SAT0-SATn, e.g., 16 or more, 50 or more, 100 or more different satellite locations, and a large number of electrodes E1-EN, e.g., 100, 200, 400, 800, etc., may provide resolution as the bioelectric signal of interest travels from one end of the nerve 104, e.g., the spine, to the other. In this manner, problems that may otherwise be encountered using a large number of wires S1 and S2, e.g., large numbers of electrodes E1-EN and one wire S1 or S2 per electrode E1-EN, may be avoided. The problems resolved by certain aspects of the method of the present invention, for example, may include electrically or communicatively connecting to the can 106, and/or problems of providing logic circuitry, amplifiers and circuitry within the can 106.

In various additional alternate aspects, one may employ software programming that would specify the direction of interest of the signal and the rate, time, how fast one expects the signal to go from one end to the other, etc., and it may then be presented to the doctor or possibly the patient.

Referring now generally to the Figures and particularly to FIG. 4A, FIG. 4A is a schematic of the exemplary first satellite SAT0, having four electrodes E1-E4 and an optional integrated circuit 110. The first electrode E1 and the second electrode E2 are electrically coupled with the first wire S1 and may transmit signals to and the can 106 via the first wire S1. The first electrode E1 and the second electrode E2 also receive electrical energy from the can 106 via the first wire S1, wherein the received electrical energy is at least partially directed toward the nerve 104. The third electrode E3 and the fourth electrode E4 are electrically coupled with the second wire S2 and may transmit signals to and the can 106 via the second wire S2. The third electrode E3 and the fourth electrode E4 also receive electrical energy from the can 104 via the second wire S2, wherein the received electrical energy is at least partially directed toward the nerve 104. The integrated circuit 110 is electrically coupled with the first wire S1 and the second wire S2 and processes instructions received from the can 106 via the first wire S1 and/or the second wire S2, whereby the integrated circuit 110 receives electrical energy from the first wire S1 and/or the second wire S2 and delivers the received electrical energy to one or more or the electrodes E1-E4 of the first satellite SAT0 in accordance with instructions received from the can 106, and optionally in accordance with preprogramming or pre-configuring of the integrated circuit 110.

When the nerve 104 is in a steady state and there is no pain signals travelling afferently, data may be taken by the lead 102 and stored in the system memory 404 (of FIG. 4B) of the can 106, and then simulate or stimulate pain, after which further data is taken by the lead 102 and stored in the can 106. Analysis of the stored data may identify what kinds of bioelectric signals have been detected passing along the nerve 104, at what pace they travel and other characteristics of interest. Another example of bioelectric signals of interest include simply pacing constant pain, e.g., as experienced by a burn patient, where one could either look for those bioelectric signals that are repetitive that represent pain or certain velocity or one could simply pace at different intervals and adjust the timing between intervals to block anything going at that rate.

FIG. 4B is a schematic of the can 106. The can 106 includes a central processing unit (CPU) 402, a system memory 404, an electrical power reservoir battery 406 (or “battery 406”), a lead interface 408, a wireless transceiver 410, an optional configurable logic 412 and a power and communications bus 414 (or “bus 414”). The bus 414 accepts electrical power from the battery 406 and provides the electrical power received from the battery 406 to the lead interface 408. The lead interface 408 accepts and/or samples electrical signals from the satellites SAT0-SATn and delivers the electrical power sourced from the battery 406 to the first wire S1 and the second wire S2 as directed by the controller 402. The system memory 404 maintains a system software (or “SW”) 416 that instructs the can 106 in performing certain aspects of the method of the present invention as disclosed herein. The wireless transceiver 410 enables the can 106 to send and receive information and instructions via radio wave and/or other suitable communications modalities known in the art.

The bus 414 both communicatively couples the circuit elements 402, 404 and 408-412 of the can 106 and provides electrical energy from the battery 406 to the circuit elements 402, 404 and 408-412

In various aspects, the method and apparatus of the present invention may interact and/or coordinate with environmental parameters such as various devices, various sources and types of data, software systems and programs, therapy programs, and pharmaceuticals. The environmental parameters may be found within the neural environment 100, external to the neural environment 100, or a combination of both. Such interactions and coordination with various environmental factors have broad applicability across various areas, including medical, financial, research and development. Such interaction and coordination may be used, inter alia, to correlate data sets, refine various analyses, provide inferences, inform decision-making, and/or enhance therapies.

More particularly, various invention parameters such as recording start time, timing interval, and neural signal characteristics, may be related to environmental parameters to bring about various results. Conversely, various invention parameters such as data set, modeling and analysis, may be used to inform decisions and/or trigger actions associated with various environmental parameters. Alternatively, various invention parameters may be coordinated with environmental parameters for multiple predetermined purposes.

Devices include, for example, implantable devices such as cardiac devices and receivers, ingestible devices, such as ingestible event markers 500 disclosed in PCT application serial no. PCT/US2006/016370 published as WO/2006/116718; PCT application serial no. PCT/US2007/082563 published as WO/2008/052136; PCT application serial no. PCT/US2007/024225 published as WO/2008/063626; PCT application serial no. PCT/US2007/022257 published as WO/2008/066617; PCT application serial no. PCT/US2008/052845 published as WO/2008/095183; PCT application serial no. PCT/US2008/053999 published as WO/2008/101107; PCT application serial no. PCT/US2008/056296 published as WO/2008/112577; PCT application serial no. PCT/US2008/056299 published as WO/2008/112578; and PCT application serial no. PCT/US2008/077753 published as WO 2009/042812 (the disclosures of which ingestible event marker applications are incorporated herein by reference); drug delivery devices such as insulin pumps; external devices such as transceivers 610 associated with the body (“body-associated signal receivers”), various computing devices such as servers, mobile devices, desktop computers, etc. Body-associated signal receivers of interest include, but are not limited to, conductively transmitted signal receivers, such as those receivers described in: PCT Application Serial No. PCT/US08/85048; PCT Application Serial No. PCT/US2007/024225 published as WO 2008/095183; PCT Application Serial No. PCT/US2007/024225 published as WO 2008/063626 and PCT Application Serial No. US2006/016370 published as WO 2006/116718; as well as U.S. Provisional Application Ser. No. 61/160,289; the disclosures of which are herein incorporated by reference.

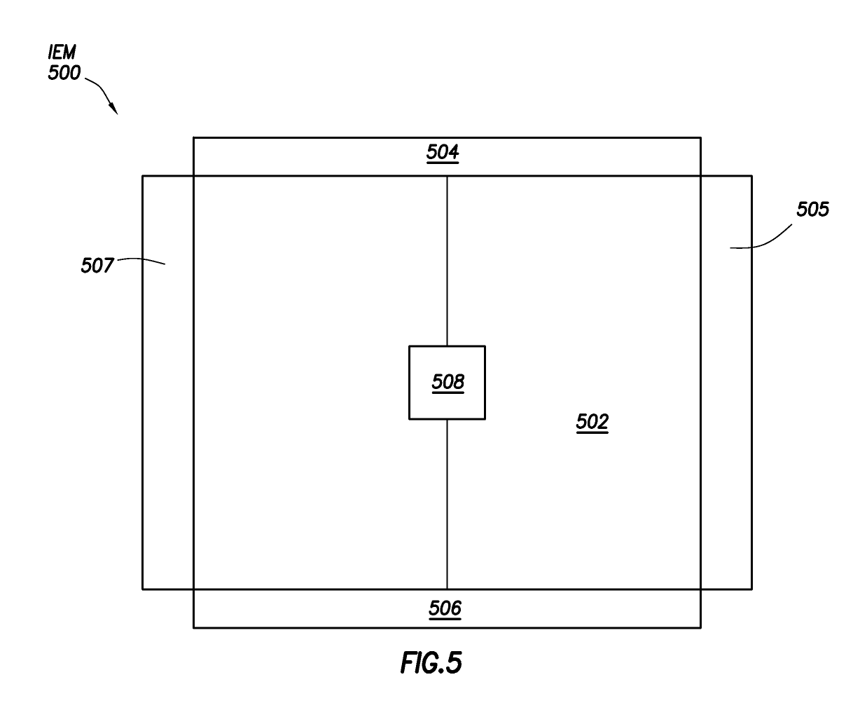

Referring now generally to the Figures and particularly to FIG. 5, FIG. 5 is a schematic of ingestible event marker (IEM) 500, as set out in U.S. patent application Ser. No. 12/564,017, filed Sep. 21, 2009, and incorporated herein in its entirety by reference. In one example, IEM 500 is combined with the pharmaceutical product, as the product or pill is ingested, the IEM 500 is activated. The IEM controls conductance to produce a unique current signature that is detected, thereby signifying that the pharmaceutical product has been taken. The IEM 500 includes a framework 502. The framework 502 is a chassis for the IEM 500 and multiple components are attached to, deposited upon, or secured to the framework 502. In this example, an ingestible material 504 is physically associated with the framework 502. The material 504 may be chemically deposited on, evaporated onto, secured to, or built-up on the framework all of which may be referred to herein as “deposit” with respect to the framework 502. The material 504 is deposited on one side of the framework 502. The materials of interest that can be used as material 504 include, but are not limited to: Cu or CuI. The material 504 is deposited by physical vapor deposition, electrodeposition, or plasma deposition, among other protocols. The material 504 may be from about 0.05 to about 500 μm thick, such as from about 5 to about 100 μm thick. The shape is controlled by shadow mask deposition, or photolithography and etching, etc. Additionally, even though only one region is shown for depositing the material, each system 500 may contain two or more electrically unique regions where the material 504 may be deposited, as desired. At a different side, for example the opposite side, another digestible material 506 is deposited, such that materials 504 and 506 are dissimilar. Although not shown, the different side selected may be the side next to the side selected for the material 504. The scope of the present invention is not limited by the side selected and the term “different side” can mean any of the multiple sides that are different from the first selected side. Furthermore, even though the shape of the system is shown as a square, the shape maybe any geometrically suitable shape. Material 504 and 506 are selected such that they produce a voltage potential difference when the IEM 500 is in contact with conducting liquid, such as body fluids. The materials of interest for material 506 include, but are not limited to: Mg, Zn, or other electronegative metals. As indicated above with respect to the material 504, the material 506 may be chemically deposited on, evaporated onto, secured to, or built-up on the framework. Also, an adhesion layer may be necessary to help the material 506 (as well as material 504 when needed) to adhere to the framework 502. Typical adhesion layers for the material 506 are Ti, TiW, Cr or similar material. Anode material and the adhesion layer may be deposited by physical vapor deposition, electrodeposition or plasma deposition. The material 506 may be from about 0.05 to about 500 μm thick, such as from about 5 to about 100 μm thick. However, the scope of the present invention is not limited by the thickness of any of the materials nor by the type of process used to deposit or secure the materials to the framework 502.

According to the disclosure set forth, the materials 504 and 506 can be any pair of materials with different electrochemical potentials. Additionally, in the aspects wherein the system 500 is used in-vivo, the materials 504 and 506 may be vitamins that can be absorbed. More specifically, the materials 504 and 506 can be made of any two materials appropriate for the environment in which the IEM 500 will be operating. For example, when used with an ingestible product, the materials 504 and 506 are any pair of materials with different electrochemical potentials that are ingestible. An illustrative example includes the instance when the IEM 500 is in contact with an ionic solution, such as stomach acids. Suitable materials are not restricted to metals, and in certain aspects the paired materials are chosen from metals and non-metals, e.g., a pair made up of a metal (such as Mg) and a salt (such as CuCl or CuI). With respect to the active electrode materials, any pairing of substances—metals, salts, or intercalation compounds—with suitably different electrochemical potentials (voltage) and low interfacial resistance are suitable.

In one aspects, one or both of the metals may be doped with a non-metal, e.g., to enhance the voltage potential created between the materials as they come into contact with a conducting liquid. Non-metals that may be used as doping agents in certain aspects include, but are not limited to: sulfur, iodine and the like. In another aspect, the materials are copper iodine (CuI) as the anode and magnesium (Mg) as the cathode.

Thus, when the IEM 500 is in contact with the conducting liquid, a current path, an example is shown in FIG. 5, is formed through the conducting liquid between material 504 and 506. A control device 508 is secured to the framework 502 and electrically coupled to the materials 504 and 506. The control device 508 includes electronic circuitry, for example control logic that is capable of controlling and altering the conductance between the materials 504 and 506.

The voltage potential created between the materials 504 and 506 provides the power for operating the system as well as produces the current flow through the conducting fluid and the system. In one aspect, the system operates in direct current mode. In an alternative aspect, the system controls the direction of the current so that the direction of current is reversed in a cyclic manner, similar to alternating current. As the system reaches the conducting fluid or the electrolyte, where the fluid or electrolyte component is provided by a physiological fluid, e.g., stomach acid, the path for current flow between the materials 504 and 506 is completed external to the IEM 500; the current path through the IEM 500 is controlled by the control device 38. Completion of the current path allows for the current to flow and in turn a receiver, not shown, can detect the presence of the current and recognize that the IEM 500 has been activate and the desired event is occurring or has occurred. In various aspects, the receiver (not shown) may be configured to contact an individual, configured to be implantable or semi-implantable, etc.

Referring again to FIG. 5, the materials 504 and 506 provide the voltage potential to activate the control device 508. Once the control device 508 is activated or powered up, the control device 508 can alter conductance between the materials 504 and 506 in a unique manner. By altering the conductance between materials 504 and 506, the control device 508 is capable of controlling the magnitude of the current through the conducting liquid that surrounds the IEM 500. This produces a unique current signature that can be detected and measured by a receiver (not shown), which can be positioned internal or external to the body. In addition to controlling the magnitude of the current path between the materials, non-conducting materials, membrane, or “skirt” are used to increase the “length” of the current path and, hence, act to boost the conductance path, as disclosed in the U.S. patent application Ser. No. 12/238,5045 entitled, “In-Body Device with Virtual Dipole Signal Amplification” filed Sep. 25, 2008, the entire content of which is incorporated herein by reference. Alternatively, throughout the disclosure herein, the terms “non-conducting material”, “membrane”, and “skirt” are interchangeably with the term “current path extender” without impacting the scope or the present aspects and the claims herein. The skirt, shown in portion at 505 and 507, respectively, may be associated with, e.g., secured to, the framework 502. Various shapes and configurations for the skirt are contemplated as within the scope of the present invention. For example, the IEM 500 may be surrounded entirely or partially by the skirt and the skirt maybe positioned along a central axis of the IEM 500 or off-center relative to a central axis. Thus, the scope of the present invention as claimed herein is not limited by the shape or size of the skirt. Furthermore, in other aspects, the materials 504 and 506 may be separated by one skirt that is positioned in any defined region between the materials 504 and 506.

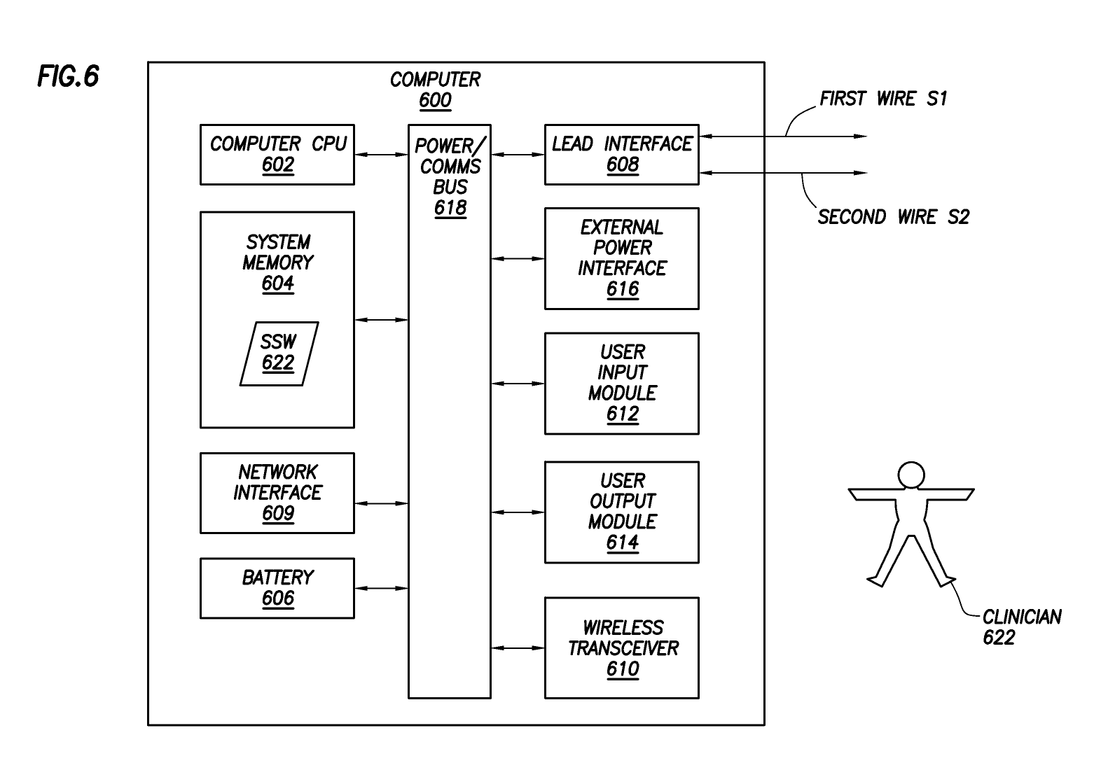

Once the control device 518 is activated or powered up, the control device 518 can alter conductance between the materials 514 and 516. Thus, the control device 518 is capable of controlling the magnitude of the current through the conducting liquid that surrounds the system 510. As indicated above with respect to IEM 500, a unique current signature that is associated with the system 510 can be detected by a receiver (not shown) to mark the activation of the system 510. In order to increase the “length” of the current path the size of the skirt 49 is altered. The longer the current path, the easier it may be for the receiver to detect the current. Referring now generally to the Figures and particularly to FIG. 6, FIG. 6 is a schematic of a computer 600 that is external to the host 110 and communicates via wireless radio communications with the can 106 and the IEM 500 via a receiver, e.g., patch 700. Computer 600 includes a computer central processing unit 602 (or “computer cpu 602”), a system memory 604, an electrical power reservoir battery 606 (or “battery 606”), a lead interface 608, a network interface 609, a wireless transceiver 610, a user input module 612, a user output module 614, an external power interface 616 and a power communications bus 618 (or “power/comms bus 618”).

The external power interface 616 is coupled with an external source of electrical power, such as an electrical power grid (not shown) or an electrical power generator (not shown). The internal bus 614 accepts electrical power from the computer battery 606 and/or the electrical power interface 616 and provides the electrical power received from the computer battery 606 and/or the external power interface 616 to the lead interface 608.

The lead interface 608 accepts and/or samples electrical signals from the satellites SAT0-SATn and delivers the electrical power to the first wire S1 and the second wire S2 as directed by the computer CPU 602.

The system memory 604 maintains a computer system software 622, or “SSW 622” that instructs the computer 600 in performing certain aspects of the method of the present invention as disclosed herein. The wireless computer transceiver 610 enables the computer 600 to send and receive information and instructions via radio wave and/or other suitable communications modalities known in the art to the can 106 and to receive information from the IEM 500.

The internal bus 618 both communicatively couples the circuit elements 602, 604 and 608-614 of the computer 600 and provides electrical energy from computer battery 606 and/or the external power interface 616 to the computer circuit elements 602, 604 and 608-614.

The user input module 612 may be or comprise a computer keyboard, a computer mouse, or other suitable computer peripheral device that enables a clinician 624 to input information and commands into the computer 600 to direct the computer 600 and/or the can 106 and lead 102 according to certain aspects of the present the invention. The user output module 614 may be or comprise a computer video monitor that displays information to the clinician 624.

The network interface 609 and/or the transceiver 610 may communicatively couple the computer 600 with an external server (not shown), a telephony network, an electronic communications network, a computer network, and/or the Internet.

To illustrate, data associated with a characteristic determined by the neural signal determination device 102, e.g., absent of desired neural signal along a path within the host 110 where presence of a neural signal would be expected, may trigger an alert communicated to the computer transceiver 610 for onward transmission either directly or indirectly to a stimulation system such as a brain or cardiac stimulation device.

Referring now generally to the Figures and particularly to FIG. 7, FIG. 7 is an illustration of interactivity of the IEM 500, the can 106, the computer 600 and the clinician 624. The computer 600 receives information from the IEM 500 via patch 700? and the can 106 via a wireless communications mode while the IEM 500 and the can 106 are positioned within the host 110. In addition, the computer 600 transmits commands, instructions and information to the can 106 via a wireless communications mode. The clinician 624 operates the computer 600 to view information related to the communication and interaction of the computer 600, the IEM 500 and the can 106, as well as to direct and control the information processing and other operations of the computer 600 and the can 106.

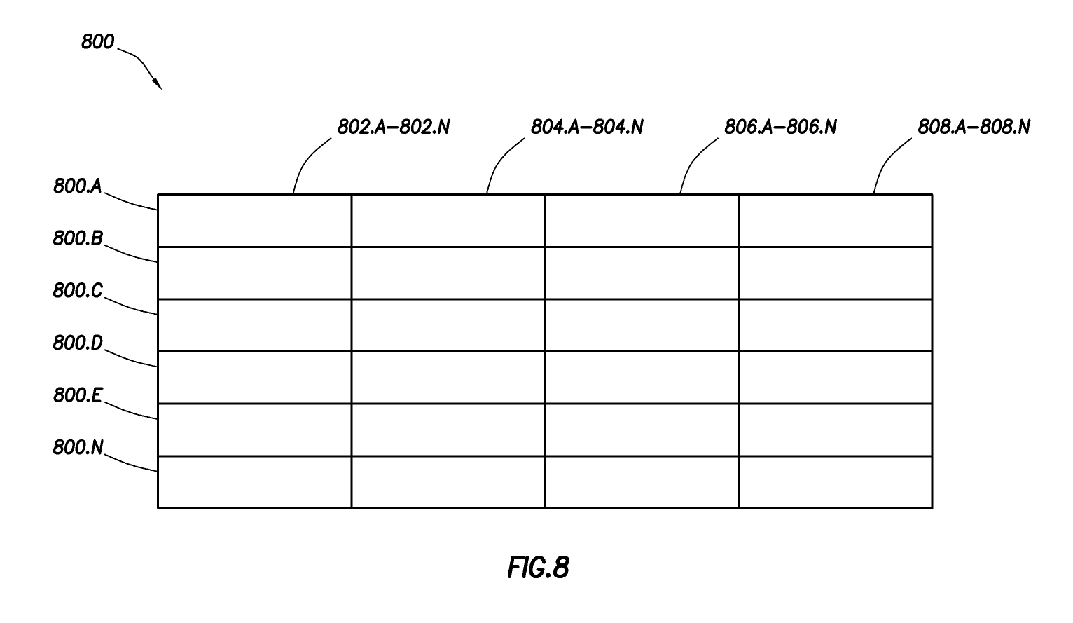

Referring now generally to the Figures and particularly to FIG. 8, FIG. 8 is a schematic of a diagnostic and therapeutic record stack 800 are stored in the system memory 404. Each diagnostic and therapeutic record 800.A-800.N (hereinafter “D&T record” 800.A-800.N) includes a record identifier 802.A-802.N, a diagnostic pattern 804.A-804.N, and a therapeutic action data 806,A-806.N. According to the method of the present invention, measurements from the electrodes E1-EN of the lead 102 are recorded in the system memory 404. These measurements are then compared by the CPU 402 to determine whether the recorded measurements match a diagnostic pattern 804 of each of the D&T records 800.A-800.N When a match is found between the recorded measurements of the lead 102 and an exemplary first diagnostic pattern 804.A of a first D&T record 800.A, the can 106 implements the associated first therapeutic action data 806.A and provides power to the electrodes E1-EN in accordance with the first therapeutic action data 806.A. In illustrative case example, the first diagnostic pattern 804.A is indicative of a bioelectric signal that induces a painful sensation to the host 110. The first therapeutic action data 806.A might therefore be a representation of instructions and data that cause the can 106 to energize the electrodes E1-EN in a pattern that nullifies or diminishes the pain inducing pulses described by the first diagnostic pattern 804.A.

The optional environmental data 808.A-808.N of each D&T record 800.A-800.N may contain information that effects the implementation by the can of the therapeutic action data 806.A-806.N.

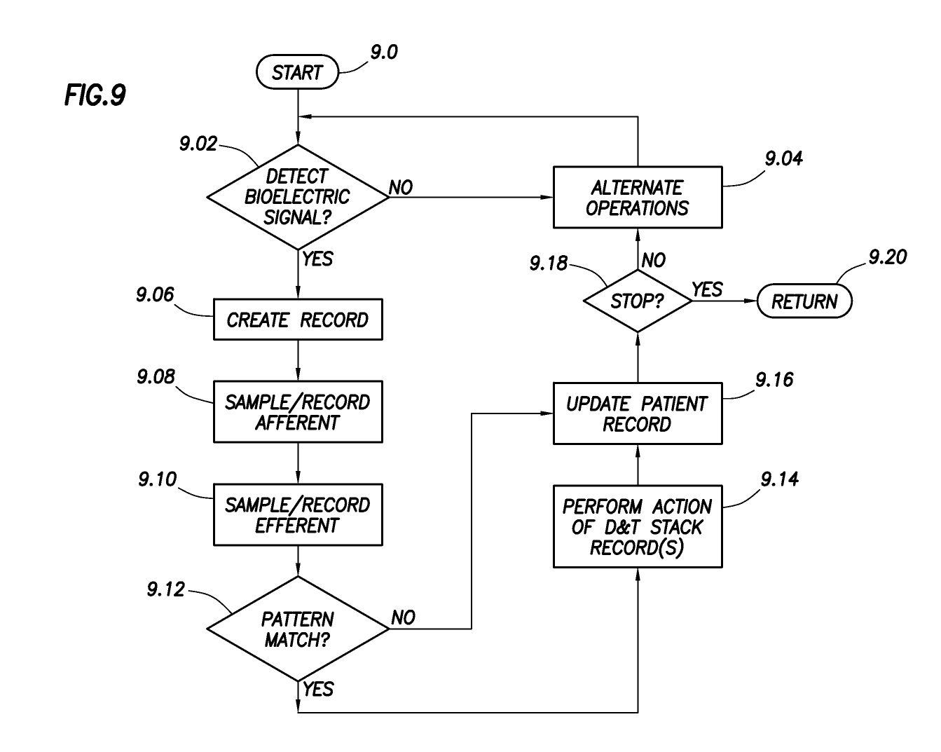

Referring now generally to the Figures and particularly to FIG. 9, FIG. 9 is a flow chart of processing and electrode excitation activity of the can 106 as directed by the can system software 416. In step 9.02 the can determines whether any electrode E1-EN has detected a bioelectric signal of sufficient electrical power to be of interest, e.g., an electrical potential greater than one millivolt in certain aspects of the method of the present invention and an electrical potential greater than one microvolt in certain alternate aspects of the method of the present invention. When no significant signal is detected by can 106 in step 9.02, the can 106 proceeds on to alternate operations in step 9.04. When a bioelectric signal of sufficient electrical power to be of interest is detected in step 9.02, the can 106 proceeds on to step 9.06 and to create a signal record. The signal record is then populated in step 9.08 with measurements taken at leads E1-EN afferent from the electrode at which the bioelectric signal was first detected, and in nearly simultaneously executed step 9.10. the signal record is populated with measurements taken at leads E1-EN efferent from the electrode at which the bioelectric signal was first detected.

The can 106 compares the signal record to each bioelectric signal signature 804.A-804.N of the D&T record stack 800 in step 9.12, and for each match found, the can 106 excites the electrodes E1-EN in accordance with the therapeutic action data 806.A-806.N associated with each D&T record 802.A-802.N matched in step 9.14. In step 9.16 the can 106 communicates the signal record to the computer 600 and informs the computer 600 of measurements and/or actions taken in steps 9.02 through 9.14, whereby the computer 600 may update a patient record stored in the computer system memory 604. The can 106 determines in step 9.18 whether to continue monitoring the electrodes E1-EN in step 9.18, and proceeds on to step 9.20 in accordance with the can system software 426. The can 106 may accept updates to the can system software 416 in step 9.04 as transmitted in wireless radio wave communications from the computer 600, to include updating of the D&T record stack 800.

The can 106 thereby, based on the characteristics of the measurements of the bioelectric signal, e.g., the magnitude of the bioelectrical parameter measurement generated by the second electrode, and a time displacement between the bioelectric signal detection of the first electrode and the generation of the bioelectrical parameter measurement by the second electrode, associate at least one bioelectric signal quality with the bioelectric signal. Other signal qualities determinable by the method of the present invention include a directionality of the bioelectric signal, a velocity of the bioelectric signal, type of the bioelectric signal, strength of the bioelectric signal, amplitude of the bioelectric signal, a pattern of the bioelectric signal, a presence of the bioelectric signal, and an absence of the bioelectric signal.

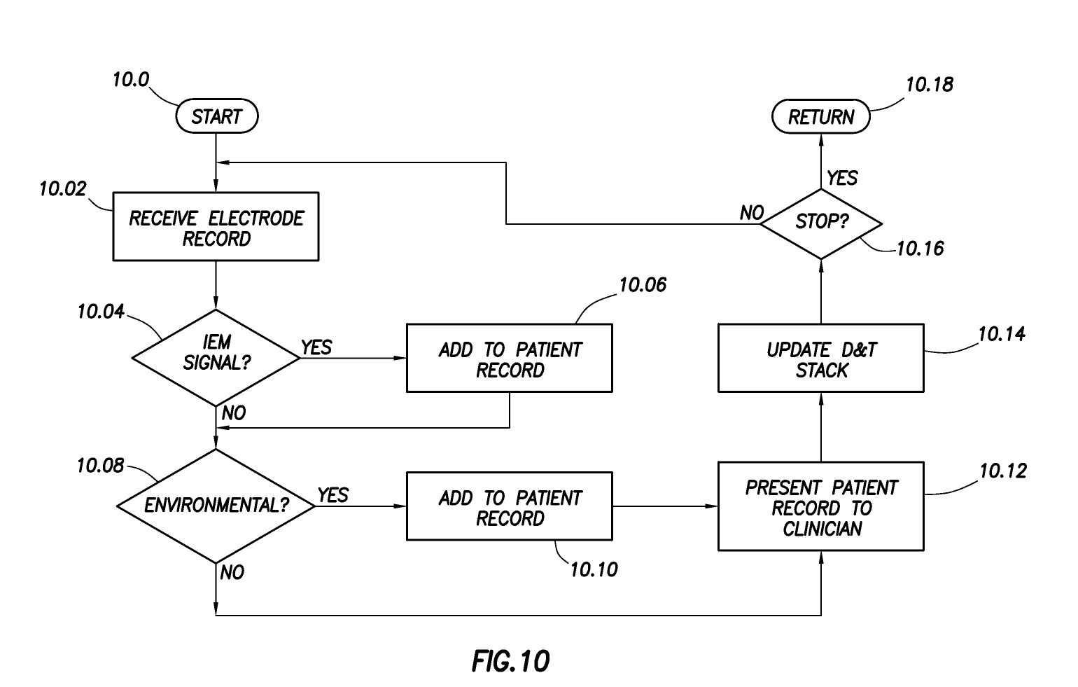

Referring now generally to the Figures and particularly to FIG. 10, FIG. 10 is a process chart of interaction of the clinician 624 and the computer 600. In step 10.02 the computer receives the signal record of steps 9.06-9.12 from the can 106. In step 10.04 the computer 600 determines whether a medication ingestion signal from the IEM 500 that is associable with the host 110 has been received, and when such a signal has been detected, the computer 600 adds a record of the signal receipt to a patient record in step 10.06, where the patient record is stored in the computer system memory 604 or accessible via the computer network interface 608. In step 10.08 the computer 600 determines whether an environmental parameter associable with the host 110 has been received via the computer input module 612 or the network interface 608, and when such an environmental parameter has been received, the computer 600 adds a record of the environmental parameter to the patient record in step 10.10. The computer 600 further updates the patient record in step 10.10 with the signal record received from the can 106 in step 10.02. In step 10.12 the patient record is presented to the clinician via the user output module 614. In step 10.14, the computer 600 receives information and commands from the clinician in step 600 and updates the patient record and the D&T record stack 800 in accordance with the clinician-provided commands and information.

The computer 600 determines in step 10.16 whether to continue monitoring for wireless communications from the can 106 and the IEM 500 in step 10.16, and optionally proceeds on to step 10.18 in accordance with the computer system software 622 and commands received from the clinician or other third party via the computer input module 612 or the network interface 608.

The clinician 624 may thus consider environmental data and ingestion marker transmissions from the IEM 500 in steps 10.12 and 10.14 to direct the activity of the can 106, the lead 102, and the computer 600.

Various sources of environmental data include, for example, data derived from various medical devices, databases, repositories, business and commercial entities, and medical organizations. Types of environmental data include, for example, data associated with healthcare regimens such as medication data, financial data, and research data.

To illustrate, data associated with characteristics of a neural signal acquired by the can 106 may be communicated to repositories for aggregation with pharmaceutical data and financial data. The aggregated neural data may be analyzed and used to inform various decisions, e.g., whether, based on neural system results, a particular pharmaceutical is a viable financial option for a particular patient population.

Various software systems and programs include, for example, systems and programs that facilitate data gathering, e.g., medical device programs, data storage and database software, and data analysis and decision support software.

Therapy programs include, for example, various healthcare regimens that include or are based on pharmaceuticals, acupuncture, physical therapy, and drug delivery.

To illustrate, a start time of recording and time intervals of recording may be correlated with acupuncture to compare and analyze neural signal characteristics and inform associated therapy enhancement adjustments based on the comparison and analysis.

In another example, a start time and timing intervals of recording may be correlated with aspects of a pharmaceutical therapy to determine, among other things, drug interaction with the neural system, and effectiveness of the therapy. Such pharmaceutical therapies include, for example, epilepsy medications, Parkinson's disease medications, cardiac therapy medications, and other pharmaceuticals related or unrelated to neural diseases and disorders.

In certain cases, a pharmaceutical 502 may be, for example, ingested with or without an ingestible event marker 500.

In cases where one or more pharmaceuticals 502 are ingested without ingestible event marker(s), recordings may be taken prior to administration of the pharmaceutical, during administration of the pharmaceutical, and/or after administration of the pharmaceutical. Recording data and characteristics may be correlated with the various times to identify therapy effectiveness.

In cases where one or more pharmaceuticals 502 are ingested with an ingestible event marker or markers 500, recordings and/or neural signal characteristics may be correlated with various pharmaceutical parameters. Such parameters include time of ingestion, which may be determined via the ingestible event marker; type of medication, which may be determined via the ingestible event marker, patient or pharmacy records, etc; dosage, which may be determined via the ingestible event marker, patient or pharmacy records.

For example, the neural signal determination device and an epilepsy therapy of the host 110 may be interactive and coordinated. The host 110 may ingest an epilepsy medication configured with an ingestible event marker. The ingestible event marker 500 may communicate data such as the time of ingestion, the type of medication, and the dosage to one or more transceivers 610 associated with the host 110. The transceiver 610, in turn, may communicate the data to a computer 600, which may trigger an alert to the transceiver 610, and onwards to the can 106, to begin recording. Recordings of bio-electrical activity measurements that indicative of patient host health characteristics may then be analyzed in view of the epilepsy therapy of the host 110 to determine therapy effectiveness and/or to optimize treatment, e.g., increase dosage, adjust ingestion intervals.

Environmental factors include a variety of parameters that may have an effect on the host 110, the host's neural system, therapy, etc. Such environmental factors include, for example, environmental temperature, time of day, activities, etc.

To illustrate, consider that triggers and precipitants for epileptic seizures may include sleep and wake cycles. The neural signal determination device 102 may be programmed or activated to coincide with sleep and wake cycles. Recorded data and characteristics may then be analyzed to gain a better understanding of patterns of triggers and/or to enhance treatment programs.

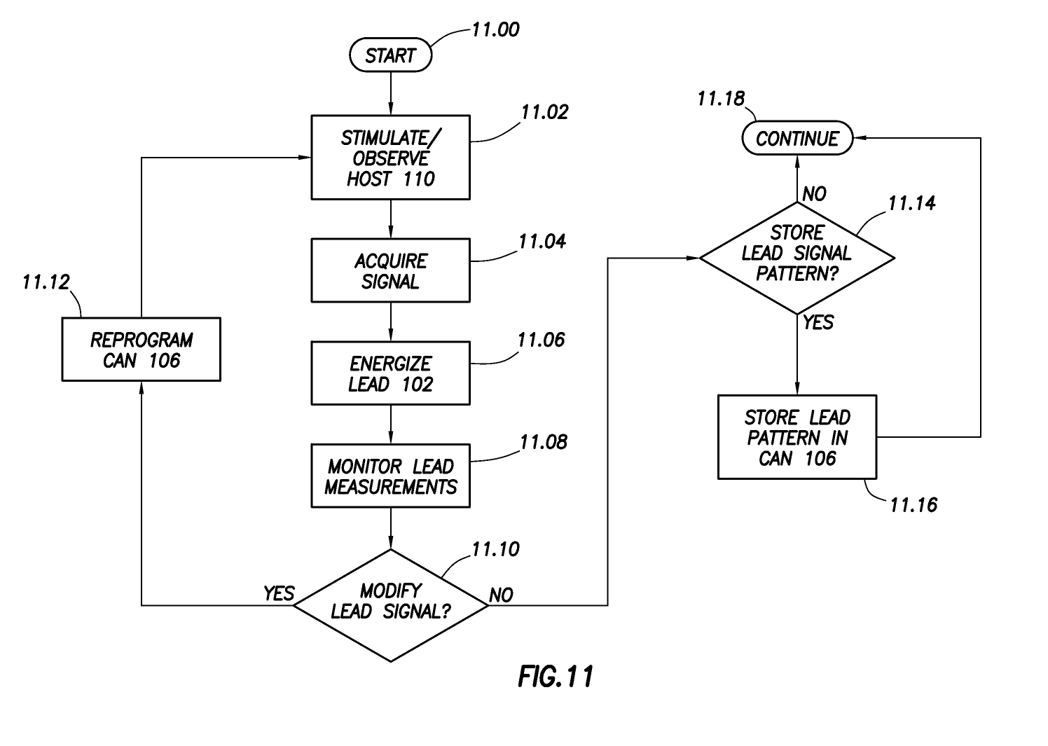

Referring now generally to the Figures and particularly to FIG. 11, FIG. 11 is a process chart of a method of modifying and storing a therapeutic action data 806.A-806.N. In step 11.02 the host is observed to have exhibited a physiological condition, e.g., experiencing a sensation of pain from a repeated bioelectric pain signal conducted by the nerve 104. Alternatively, the clinician 624 may in step 11.02 impose or cause the physiological condition to occur or be maintained, e.g., by sustained jabbing of a nerve-ending of the nerve 104. The electrodes E1-EN of the lead 102 sense the bioelectric signal generated by or causing the physiological condition step 11.02, and the electrode measurements are provided to the controller 106. The lead measurements of step 11.04 may optionally be transmitted to the computer 600 for observation by the clinician 624. One or more leads E1-EN are energized in step 11.06 in accordance with a therapeutic action plan *06.A-806.N selected from the D&T record stack 800, wherein the D&T record stack may be located within the can 106 and/or the computer 600. When the selected therapeutic action plan 806.A-806.N is sourced in step 11.06 from the computer 600, the selected therapeutic action plan 806.A-806.N is first wirelessly transmitted from the computer 600 to the can 106. Measurements of the electrodes E1-EN taken from the nerve 104 are received by the can 106 in step 11.08 while the lead 102 is being energized in accordance with the selected therapeutic action plan 806.A-806.N, and these updated measurements of the electrical state of the nerve 104 are transmitted to the computer 600 for observation be the clinician 600. The can 106 determines in step 11.10, optionally as directed by one or more commands from the computer 600, whether to cease energizing the lead 102. When the can 106 determines in step 11.10 to continue energizing the lead 102, the can 106 accepts a modified therapeutic action plan 806.A-806.N in step 11.12 as provided by the computer 600, and energizes the lead 102 in accordance with the received modified the selected therapeutic action plan 806.A-806.N in that same step 11.12. It is understood that the computer system software 622 enables the clinician 624 to direct the computer 600 to generate, modify and/or cause one or more selected therapeutic action plans 806.A-806.N to be transmitted to the can 106. The can 106 proceeds on from step 11.12 to execute another series of commands 11.02-11.10.

When the can 106 determines in step 11.10 to cease energizing the lead 102, the can 106 proceeds on to step 11.14.

The clinician optionally directs the computer 600, and the can 104 by means of wireless communications from the can 106, in step 11.14 to store a modified pattern of electrode energizing instructions, as applied in at least one execution cycle of step 11.12, as a therapeutic action plan 806.A-806.N in the D&T record stack 800. In accordance with clinician instructions, the can 106 and the computer 600 stores the modified therapeutic action plan 806.A-806.N in step 11.16 as selected by the clinician in a D&T record 800.A-800.N, wherein the instant D&T record 800.A-800.N includes a digitized representation of the bioelectric signal acquired in step 11.04.

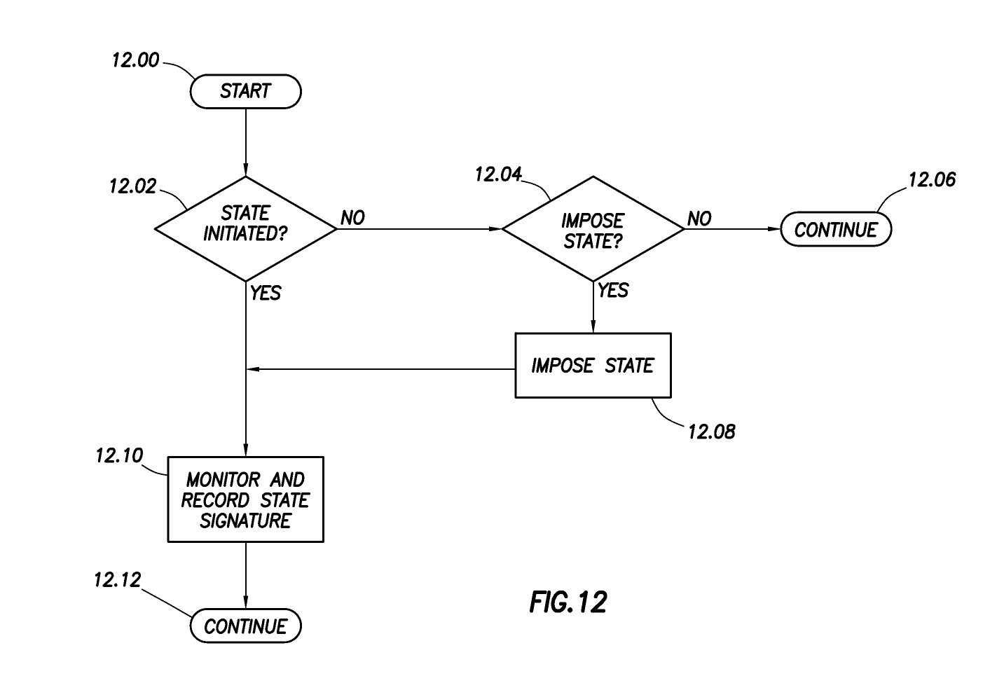

Referring now generally to the Figures and particularly to FIG. 12, FIG. 12 is a process chart of a method of identifying and recording a digitized representation of a bioelectric energy state of the host 110. The clinician 624 determines in step 12.02 whether the bioelectric energy state of the host 110 is being exhibited by the host 110. When the clinician determines in step 12.02 that the bioelectric state-of-interest is not being instantiated by the host 110, the clinician may proceed to step 12.04 to determine whether the bioelectric energy state shall be imposed, and optionally elect to impose the bioelectric energy state in step 12.08 by affecting the state of the host 110.

The bioelectric energy state is monitored by the lead 102 in step 12.10 and a digitized representation of the measurements of the electrodes E1-EN is stored as a state signature in the can 106 and/or the computer 600 in the same state 12.10.

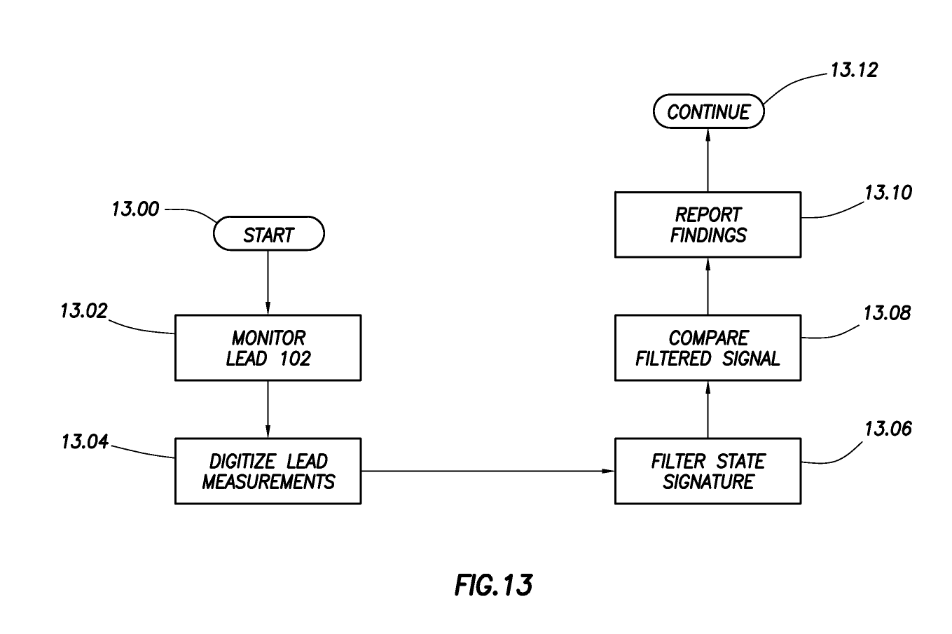

Referring now generally to the Figures and particularly to FIG. 13, FIG. 13 is a process chart of a method of improving the measurement and detection of an additional bioelectric signal that occurs simultaneously with the bioelectric energy state of step 12.10 of FIG. 12. The can 106 accepts measurements from the lead 102 in step 13.02 and transfers these measurements of the electrode E1-EN to the computer 600. The computer 600 digitizes the measurements in step 13.04, and in step 13.06 applies the state signature of step 12.10 of FIG. 12 to filter out the contribution of the additional bioelectric signal to the lead measurements of step 13.02. The computer 600 compares the resultant filtered measurements generated in step 13.06 to the D&T record stack 800 in step 13.08. In step 13.10 the computer 600 reports the results of the comparison of step 13.10 of the filtered measurements of step 13.08 to the clinician via the user output module 614 and/or via the network interface 609.

One skilled in the art will recognize that the foregoing examples are not to be taken in a limiting sense and are simply illustrative of at least some of the aspects of the present invention.

What is claimed is:

1. A method of monitoring bioelectric activity of a living being with at least two electrodes configured to be implanted in the living being, the method comprising:
a. detecting and measuring a bioelectric signal by a first electrode;
b. generating a bioelectrical parameter measurement by at least a second electrode; and
c. based on the magnitude of the bioelectrical parameter measurement generated by the second electrode, and a time displacement between the bioelectric signal detection of the first electrode and the generation of the bioelectrical parameter measurement by the second electrode, associating at least one bioelectric signal quality with the bioelectric signal.

2. The method of claim 1, further comprising:
e. receiving an instruction by at least one electrode of the at least two electrodes; and
f. based on the instruction, generating an electrical signal by the at least one electrode,
wherein the electrical signal affects a nerve of the living being.

3. The method of claim 1, wherein the bioelectric signal quality is selected from the quality group consisting essentially of directionality of the signal, velocity of the signal, type of the signal, strength of the signal, amplitude of the signal, pattern of the signal, presence of the signal, and absence of the signal.

4. The method of claim 1, further comprising deriving a therapeutic recommendation at least partly on the basis of the associated bioelectric signal quality.

5. The method of claim 4, further comprising:
e. receiving an instruction based on the therapeutic recommendation by at least one electrode of the at least two electrodes; and
f. based on the instruction, generating an electrical signal by the at least one electrode,
wherein the electrical signal affects the nerve to facilitate a therapeutic treatment associated with the therapeutic recommendation.

6. A neural signal determination device comprising:
at least two conductors; and
at least two electrodes located at respective electrode locations associated with at least a portion of a nerve, each electrode of the at least two electrodes electrically coupled with at least one conductor of the at least two conductors,
wherein each electrode of the at least two electrodes at a predetermined time interval, detects information associated with a signal travelling along at least a portion of the nerve and transmits the information along at least one conductor of the at least two conductors.

7. The neural signal determination device of claim 6, further comprising a controller, the controller communicatively coupled with the at least two conductors, wherein the controller receives the information transmitted from each electrode.

8. The neural signal determination device of claim 7, wherein the controller comprises an electrical power source controllably coupled with at least one conductor of the at least two conductors, and the controller selectively directs electrical power through the at least one conductor of the at least two conductors, wherein electrical power is directed through at least one electrode and at least partly toward the nerve.

9. A method comprising:
a. detecting and measuring a bioelectric signal associated with a first nerve location of a living being by a first electrode of a plurality of electrodes;
b. measuring the bioelectric signal associated with a second nerve location of the living being by a second electrode of the plurality of electrodes at a predetermined time interval; and
c. at least partly on the basis of the detection of the bioelectric signal by the first electrode and the measurement by the second electrode at the predetermined time interval after the bioelectric signal detection, associating at least one bioelectric signal quality with the bioelectric signal.

10. The method of claim 9, further comprising correlating the assigned bioelectric signal quality with at least one environmental parameter of an aspect of the living being.

11. The method of claim 10, wherein the at least one environmental parameter is selected from the parametric group consisting essentially of a pharmaceutical introduction into the living being, an ingestion of a substance by the living being, a medical quality of the living being, and a behavior of the living being.

12. The method of claim, wherein the bioelectric signal quality is selected from the quality group consisting essentially of directionality of the signal, velocity of the signal, type of the signal, strength of the signal, amplitude of the signal, pattern of the signal, presence of the signal, and absence of the signal.

13. The method of claim 12, further comprising directing electrical power through at least one electrode and at least partly toward the nerve.

14. A method of monitoring, with a plurality of implantable electrodes, bioelectric activity associated with a nerve of a living being with a plurality of implanted electrodes, the method comprising:
a. at least partly on a basis of an environmental parameter, measuring a bioelectric signal by each electrode of the plurality of implanted electrodes at a predetermined time interval; and
b. at least partly on the basis of the measurements of the bioelectric signal by the plurality of implanted electrodes, associating at least one bioelectric signal quality with the bioelectric signal.

15. The method of claim 14, wherein the environmental parameter is selected from the parametric group consisting essentially of a pharmaceutical introduction into the living being, an ingestion of a substance by the living being, a medical quality of the living being, and a behavior of the living being.

16. The method of claim 14, wherein the bioelectric signal quality is selected from the quality group consisting essentially of directionality of the signal, velocity of the signal, type of the signal, strength of the signal, amplitude of the signal, pattern of the signal, presence of the signal, and absence of the signal.

17. The method of claim 14, further comprising generating an electrical signal by the at least one electrode, wherein the electrical signal affects the nerve.

18. A method comprising:
a. applying a nullifying signal through a plurality of electrodes and at least partly toward a nerve of a living being;
b. monitoring the resultant electrical behavior of the nerve;
c. applying a modified nullifying signal via the plurality of electrodes to the nerve;
d. monitoring the resultant electrical behavior of the nerve under the influence of the modified nullifying signal;
e. storing a record of the modified nullifying signal in a controller; and
f. regenerating the modified nullified signal via the plurality of electrodes as directed by the controller, whereby an improved reduction or nullifying of the bioelectric signal is achieved.

19. The method of claim 18, wherein the series of similar bioelectric signals are intentionally induced to enable the storing of the record of the modified nullifying signal.

20. A method of monitoring bioelectric activity of a living being with a plurality of electrodes, the method comprising:
a. detecting a bioelectric state signal by the plurality of electrodes as determined by a communicatively coupled the controller;
b. recording a digitized representation of the bioelectric state signal in the controller;
c. detecting a second bioelectric signal by the plurality of electrodes as determined by the controller;
d. forming a digitized representation of the second bioelectric signal; and
e. filtering the digitized representation of the bioelectric state signal from the digitized representation of the second bioelectric signal, whereby the contribution of bioelectric activity contributed by the state of the living being is removed from an analysis of the second bioelectric signal.
